(19) United States
(12) Patent Application Publication
(10) Pub. No.: US 2011/0236771 A1
(43) Pub. Date: Sep. 29, 2011

(54) MUTANT GLUCONATE DEHYDROGENASE

(75) Inventors: Daisuke Yamaguchi, Kanagawa (JP); Yoshio Goto, Kanagawa (JP); Seiji Yamada, Kanagawa (JP); Yuichi Tokita, Kanagawa (JP)

(73) Assignee: SONY CORPORATION, Tokyo (JP)

(21) Appl. No.: 13/132,257

(22) PCT Filed: Nov. 30, 2009
(86) PCT No.: PCT/JP2009/070100

§ 371 Date: Jun. 1, 2011

(30) Foreign Application Priority Data
Dec. 1, 2008 (JP) 2008-306448
Oct. 7, 2009 (JP) 2009-233466

(51) Int. Cl. C12N 9/04; H01M 8/16

(52) U.S. Cl. 429/401; 435/190

(57) ABSTRACT

The present disclosure provides a mutant gluconate dehydrogenase whose enzyme activity and/or heat resistance is a predetermined level or higher. The mutant gluconate dehydrogenase is made by an amino acid sequence obtained by deleting, substituting, adding or inserting one or plural amino acids in an amino acid sequence represented by sequence number 1. The mutant gluconate dehydrogenase displays enzyme activity which is equal to or higher than 120% of a wild-type gluconate dehydrogenase made by an amino acid sequence represented by sequence number 1, and/or displays residual enzyme activity after heat treatment under predetermined conditions, which is equal to or higher than 20% of the enzyme activity before the heat treatment.

### CROSS REFERENCE TO RELATED APPLICATIONS

The present application is a National Stage of International Application No. PCT/JP2009/070100 filed on Nov. 30, 2009, which claims priority to Japanese Patent Application No. 2009-233466 filed in the Japanese Patent Office on Oct. 7, 2009, and Japanese Patent Application No. 2008-306448 filed in the Japanese Patent Office on Dec. 1, 2008, the entire contents of which are being incorporated herein by reference.

### SEQUENCE LISTING

This disclosure includes a sequence listing submitted as a text file pursuant to 37 C.F.R. §1.52(e)(v) named CI-#9212263-v1-Sequence_Listing_ST25.TXT, created on May 31, 2011, with a size of 235,555 bytes, which is incorporated herein by reference. The attached sequence descriptions and Sequence Listing comply with the rules governing nucleotide and/or amino acid sequence disclosures in patent applications as set forth in 37 C.F.R. §§1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984). The symbols and format used for nucleotide and amino acid sequence data comply with the rules set forth in 37 C.F.R. §1.822.

### BACKGROUND

Enzymes are catalysts in living organisms, which smoothly catalyze a number of reactions related to maintain the life under mild conditions in the living organisms. The enzyme performs metabolic turnover in a living organism, is produced as necessary in the living organism, and displays its catalytic function.

At present, techniques for using enzymes on the outside of a living organism are already practically used or examined for practical use. For example, the techniques using enzymes are being developed in various technical fields such as production of useful materials, production, measurement, or analysis of energy-related materials, environment preservation, medical services, and the like. In relatively recent years, techniques of an enzyme battery (see, for example, Patent document 1) as a kind of a fuel cell, an enzyme electrode, an enzyme sensor (sensor for measuring a chemical material by using an enzyme reaction), and the like are also proposed.

In the technique of fuel cells, development is being made from a fuel cell using a combustible material such as methanol or hydrogen as a fuel in the past to a fuel cell using a safer compound such as glucose. However, with respect to a high-reactive compound having a relatively simple structure such as methanol or hydrogen, a metal catalyst such as platinum acts effectively, and with respect to a low-reactive compound having a small risk and low toxicity such as glucose, the catalyst efficiency of a metal catalyst is extremely low. Consequently, as described above, an enzyme battery using enzyme as a catalyst is being proposed. Enzymes have high catalytic capability and excellent substrate specificity and stereoselectivity, so that they efficiently catalyze even a compound with a low-reactive reaction such as glucose. Therefore, by including enzyme in an electrode of a fuel cell, a safe fuel cell is allowed to be realized.

In the case of using enzyme outside of a living organism, it is important that the activity of enzyme is high and enzyme reaction speed is high. In addition, high stability to change in environment and high continuousness of activity is also necessary. However, since the chemical body of enzyme is protein, there are drawbacks such that enzyme is easily denatured by heat, pH, and the like, and the stability on the outside of a living organism is lower than that of the other chemical catalysts such as a metal catalyst. To solve the drawbacks, Patent documents 1 and 2 disclose enzyme variants (diaphorase) whose activity and heat resistance is increased to a predetermined level or higher by preparing mutant-type protein by artificially altering the base sequence of a gene which codes protein.

### CITATION LIST

Patent Documents
Patent Document 1: Japanese Unexamined Patent Application Publication No. 2004-71559
Patent Document 2: Japanese Unexamined Patent Application Publication No. 2007-143493
Patent Document 3: Japanese Unexamined Patent Application Publication No. 2008-48703

### SUMMARY

The present invention disclosure relates to a mutant gluconate dehydrogenase. More particularly, the disclosure relates to a mutant gluconate dehydrogenase whose enzymatic activity and/or heat resistance is a predetermined level or higher.

“Gluconate5-dehydrogenase: Gn5DH” is enzyme which takes electrons from gluconic acid and gives them to NAD to catalyze the reaction of generating NADH. In an enzyme battery, the Gn5DH takes electrons from gluconic acid which is generated by oxidizing glucose by gluconate dehydrogenase and gives them to NAD to catalyze the reaction of generating NADH.

It is a main object of the present disclosure to provide mutant gluconate dehydrogenase whose enzyme activity and/or heat resistance is increased to a predetermined level or higher with a view to wider applicability of gluconate dehydrogenase on the outside of a living organism.

To solve the above-mentioned problems, the present disclosure provides a mutant gluconate dehydrogenase made by an amino acid sequence obtained by deleting, substituting, adding or inserting one or plural amino acids in an amino acid sequence represented by sequence number 1, the mutant gluconate dehydrogenase displaying enzyme activity which is equal to or higher than 120% of that of a wild-type gluconate dehydrogenase made by an amino acid sequence represented by sequence number 1.

As such a mutant gluconate dehydrogenase, a mutant gluconate dehydrogenase represented in sequence number 19 or 20 is provided.

The present disclosure also provides a mutant gluconate dehydrogenase displaying enzyme activity which is equal to or higher than 120% of that of a wild-type gluconate dehydrogenase, and having residual enzyme activity after heat treatment performed at 47.5° C. for 10 minutes, which is equal to or higher than 20% of the enzyme activity before the heat treatment.

As such a mutant gluconate dehydrogenase, a mutant gluconate dehydrogenase represented in any of sequence numbers 2 to 6 is provided.

Further, the present disclosure provides a mutant gluconate dehydrogenase displaying enzyme activity which is equal to or higher than 120% of that of a wild-type gluconate dehydrogenase, and having residual enzyme activity after heat treatment performed at 53° C. for 10 minutes, which is equal to or higher than 20% of the enzyme activity before the heat treatment.

As such a mutant gluconate dehydrogenase, a mutant gluconate dehydrogenase represented in any of sequence numbers 22 to 29, 31 to 41, and 42 is provided.

In addition, the present disclosure provides a mutant gluconate dehydrogenase displaying enzyme activity which is equal to or higher than 120% of that of a wild-type gluconate dehydrogenase, and having residual enzyme activity after heat treatment performed at 57.5° C. for 10 minutes, which is equal to or higher than 20% of the enzyme activity before the heat treatment.

As such a mutant gluconate dehydrogenase, a mutant gluconate dehydrogenase represented in any of sequence numbers 70 to 76 is provided.

The present disclosure also provides a mutant gluconate dehydrogenase represented in any of sequence numbers 11 to 18 as a mutant gluconate dehydrogenase having residual enzyme activity after heat treatment performed at 47.5° C. for 10 minutes, which is equal to or higher than 20% of the enzyme activity before the heat treatment.

The present disclosure also provides a mutant gluconate dehydrogenase represented in any of sequence numbers 51 to 64 and 66 as a mutant gluconate dehydrogenase having residual enzyme activity after heat treatment performed at 53° C. for 10 minutes, which is equal to or higher than 20% of the enzyme activity before the heat treatment.

Further, the present disclosure provides a mutant gluconate dehydrogenase represented in any of sequence numbers 77 to 106 as a mutant gluconate dehydrogenase having residual enzyme activity after heat treatment performed at 57.5° C. for 10 minutes, which is equal to or higher than 20% of the enzyme activity before the heat treatment.

y the present disclosure, a mutant gluconate dehydrogenase whose enzyme activity and/or heat resistance is equal to or higher than a predetermined level is provided.

### DETAILED DESCRIPTION

Mutant gluconate dehydrogenases according to the present disclosure have enzyme activity and/or heat resistance higher than that of wild-type mutant gluconate dehydrogenases made by an amino acid sequence represented by sequence number 1. Therefore, by using the mutant gluconate dehydrogenases, high enzyme reaction speed and activity continuousness are obtained in the techniques such as production of useful materials, production, measurement, or analysis of energy-related materials, environment preservation, medical services, an electrochemical device, and the like. In particular, by including the mutant gluconate dehydrogenases in a fuel electrode of an enzyme battery, an enzyme battery continuously displaying high output can be obtained.

### EXAMPLES

### Example 1

### 1. Cloning, Expression, and Purification of Gluconate dehydrogenase (Gn5DH) Gene Derived from Escherichia coli K12

(1-1) Isolation of Genomic DNA from Escherichia coli K12 and Purification

Escherichia coli K12 is a strain of Escherichia coli widely used as the host of a recombinant DNA experiment. With respect to the Escherichia coli K12, a chromosome map which is the most specific in the living world is clarified. Escherichia coli K12 was cultured in accordance with the law and harvested by centrifugal separation, and a genome DNA was isolated by using a Wizard Genomic DNA purification kit (Promega K.K.) (refer to the instruction manual that comes with the product for the details of the method).

(1-2) Cloning of Gn5DH

A Gn5DH gene was amplified from the obtained genome DNA by PCR. The Gn5DH gene of Escherichia coli K12 is registered as accession number NC_{—}000913 [REGION: complement (4490610 . . . 4491374)] (refer to sequence number 67) in the Nucleotide database of NSBI (http://www.ncbi.nlm.nih.gov/sites/entrez? db=nuccore&itool=toolbar).

As a DNA polymerase, a Pfu DNA polymerase (Stratagene Corp.) was used, and primers having the sequences as illustrated in Table 1 were used. In addition, the underlines indicate the Nde1 sequence (Forward primer) and the BamH I sequence (Reverse primer).

TABLE 1

| | | |
|---|---|---|
| Forward | 5′-ggaattccat atgaacgatc | Sequence |
| primer | tattttcact g-3′ | number 68 |
| Reverse | 5′-gcggatcctt aaacagccac | Sequence |
| primer | taacatgc-3′ | number 69 |

A PCR product of a Gn5DH gene was purified with the PCR Cleanup kit (manufactured by Qiagen K.K.) and the resultant was confirmed by agarose electrophoresis. The base sequence was confirmed by a DNA sequencer.

(1-3) Introduction of Gn5DH Gene to Vector

An amplified fragment of the Gn5DH gene was processed with BamHI and Nde1 and purified by using the PCR cleanup kit (Qiagen K.K.). A vector pET12a (Novagen, Merck KGaA) was processed with BamHI and Nde1 and similarly purified. The two kinds of fragments were ligated by T4ligase, an XL1-blue electrocompetent cell (Stratagene Corp.) was transformed by a product, and cultured on an LB-amp plate, and production was increased.

An obtained plasmid was treated with BssHII, insertion of the Gn5DH gene was confirmed by agarose electrophoresis, and the base sequence was analyzed.

(1-4) Transformation of Escherichia Coli

The plasmid was introduced in E. coli BL21 (DE3) (Novagen, Merck KGaA) by heat shock to perform transformation. The resultant was subjected to prior culture in SOC for one hour at 37° C. and grown in an LB-amp agar medium, a part of the colony was fluid-cultured, and expression of Gn5DH was confirmed by SDS-PAGE. 3 mL of a transformant culture was separated by centrifugation, a 2×YT culture medium was added to an E. coli pellet, and the resultant was dispersed and stored at −80° C.

(1-5) Mass Culture and Protein Purification

A frozen sample of the transformant was grown in the LB-amp agar medium, and a colony was picked up and subjected to prior culture until OD600 becomes about 1 in 100 mL of LB-amp. The resultant was grown in 18 mL of the LB-amp and shaking-cultured at 37° C. until OD600 was saturated at about 2. Fungus bodies were collected from the culture by centrifugal separation (5 kG) (20g in yield in wet condition). A fungus body pellet was frozen at −80° C. and melted, after that, bacteriolyzed in solution of 200 mL of 50 mM Tris-HCl, ph 8.0, 1 mM EDTA, 1 mM DTT, and 1 mM PMSF at 0° C. by ultrasonic process, and a solution fraction was collected by centrifugal separation (9.5 kG).

The solution was put on an anion-exchange column (Sepharose QFastFlow, Amersham Bioscience), and a fraction containing Gn5DH was collected and condensed by ultrafiltration (solution amount: 20 mL, Centriplus centrifugal filter unit YM-30, Millipore). Subsequently, the sample was put in a gel filtration column (Sephacryl S-200, Amersham Bioscience) and a fraction containing Gn5DH was collected.

### Example 2

### 2. Generation of Mutant Library by Random Mutation of Gn5DH Gene and Screening of High-Active High-Heat-Resistive Mutant (First Generation)

(2-1) Error-Prone PCR by GeneMorph (registered trademark)

A gene library of a Gn5DH mutant was generated by the Error-prone PCR, and the gene was introduced in a vector DNA, and made expressed in E. coli. The “Error-prone PCR” is a method of making replicated DNA fragments undergo mutation by using the phenomenon that DNA polymerase introduces random copying errors in the base sequence at the time of DNA fragment replication reaction by PCR. Although various methods are reported, GeneMorph (registered trademark) of Stratagene Corp. was selected from products. As the template DNA, the above-described plasmid in which the Gn5DH gene of E. coli K-12 is introduced was used. The primer used for cloning of the gene was used. The PCR was conducted according to the manual of GeneMorph (registered trademark).

(2-2) Introduction of Gn5DH Gene to Vector

The error-prone PCR product was subjected to restriction enzyme process with Nde I and BamH I. A reaction was conducted at 37° C. for two hours and, after that, a reaction product was purified by using the Qiaquick PCR purification kit (Qiagen K.K.). On the other hand, like the PCR product, a vector pET12a was similarly subjected to restriction enzyme process with Nde I and BamH I (at 37° C. for two hours).

The reaction products of the restriction enzyme process were separated by low-melting agarose gel electrophoresis, and a corresponding open circular vector DNA was purified by using a Qiaquick Gel Extraction Kit (Qiagen K.K.). Subsequently, by treating the restriction enzyme process product of the purified vector with alkaline phosphatase, 5′ terminal was dephosphorylated. A reaction product was purified by a Qiaquick PCR purification Kit (Qiagen K.K.). The error-prone PCR product (that is, Gn5DH mutant gene library) obtained as described above was ligated to a vector subjected to the restriction enzyme/dephosphorylation. For the ligation reaction, Ligation Kit Mighty Mix (Takara Bio Inc.) was used. The reaction product was purified by ethanol precipitation.

(2-3) Generation and Transformation of Competent Cell

As a competent cell, an electrocompetent cell of BL21 (DE3) prepared by the inventors was used. 40 μL of a competent cell frozen sample was melted on the ice, 0.5 μL of a DNA sample having a concentration of about 1 μg/μL was mixed, and all of the resultant was set in an electroporation cuvette having a 0.1 cm gap size, and was transformed by application of voltage of 1800 kV. 960 μL of an SOC medium was added, and the resultant was shaken as prior culture for one hour at 37° C. 5 to 50 μL of the culture fluid was inoculated to an LB-amp agar medium, and incubation was performed overnight at 37° C.

(2-4) Screening

Single colonies on the agar medium were inoculated to LB-amp fluid media (150 μL) in a 96-well plate with a pick. Two wells were allocated for E. coli which produces wild types. The top face of the well plate was sealed with a gas permeable adhesive sheet (ABgene) and also covered with an accompanying cover, and shaking culture was performed overnight (up to 14 hours) at 37° C. 50 μL each of the culture solution was mixed well with 15 μL of Bug Buster (Novagen, Merck KgaA) by pipetting on a new well plate. The well plate was covered, and the mixture was bacteriolyzed by incubation for 30 minutes at 25° C. Subsequently, 75 μL of 0.1M Tris-HCl, pH8.0 and 10 μL of 0.1 M NAD were added at room temperature. At this time, one of the wild-type samples was taken as a non-heated control sample to a microtube and left at room temperature. A plate was sealed with a commercially-available OPP tape, and heat treatment was performed at 50° C. for 35 minutes (in a thermostat chamber). The plate was left still and cooled to room temperature, and the wild-type sample which was put aside was put again on the plate. 10 μL of 0.5 M sodium gluconate solution and 1 μL of 10 g/L diaphorase solution were added to each sample and, further, 5 μL of 20 mM anthraquinone sulfonic acid (AQS) 20 DMSO solution was sequentially added. The plate was sealed with an OPP tape and the solutions were stirred by a vortex mixer for five seconds. Color development states were recorded by a camera. Samples displaying stronger color generation by reduction of the AQS as compared with the wild-type samples were selected as heat-resistant candidates. With respect to the samples passed through the screening, a part of the culture remaining on the 96-well plate was inoculated to 4.5 mL of an LB culture medium and cultured overnight to purify plasmid and refrigerated, and further, inoculated to 4 mL of an LB culture medium and cultured until O.D.600 becomes about 0.4. The samples were harvested by centrifugal separation, suspended in 2 mL of a 2× YT culture medium, frozen by liquid nitrogen, and stored at −80° C.

(2-5) Abundant Expression and Purification of Gn5DH Mutant Each of the samples in the 96-well plate was mass-cultured by the method described in Example 1, and the Gn5DH mutant was purified.

(2-6) Enzyme Activity Evaluation Test

The enzyme activity of the purified Gn5DH was evaluated by detecting NADH generated by reaction between gluconate and NAD at 25° C. by using absorbance of 340 nm (ε340=6.3×103 M−1cm−1). A reaction was started by injecting 1 mL of 100 mM Tris-HCl, pH 8.0, 2 mM NAD, and 10 mM gluconate solution in a UV cell (optical path length of 10 cm and volume of 1 mL) and adding Gn5DH to the solution. The activity was determined on the basis of a tilt of a region in which light absorption immediately after the start changes linearly. The concentration of Gn5DH was determined on the basis of a standard curve generated using bovine serum albumin as a standard by the Bradford method.

(2-7) Heat Resistance Test

After performing heat treatment on the enzyme solution, the above enzyme activity evaluation test was conducted and the enzyme activity which remains after the heat treatment (“residual enzyme activity”). The heat treatment was conducted by heating the enzyme solution by an aluminum block heater at 47.5° C. for 10 minutes, 53° C. for 10 minutes, or 57.5° C. for 10 minutes.

(2-8) Result

Table 2 illustrates Gn5DH mutants displaying higher activity or heat resistance than the wild-type Gn5DH as a result of the enzyme activity evaluation test and the heat resistance test (process at 47.5° C. for ten minutes). In the table, the “enzyme activity residual ratio” denotes the residual activity after the heat treatment in percentage, obtained by performing the activity evaluation test under the same conditions before and after the heat treatment.]

TABLE 2

| | | | | Enzyme |
|---|---|---|---|---|
| | | | Residual | activity |
| Sequence | Kinds of | Enzyme | enzyme | residual |
| number | mutants | activity | activity | ratio |
| 1 | WT | 6.22 | 0.77 | 12.4 |
| 2 | I155T | 12.39 | 10.87 | 87.7 |
| 3 | A63V/V124I | 11.05 | 3.45 | 31.2 |
| 4 | V254L | 9.7 | 8.31 | 85.7 |
| 5 | T154S | 8.95 | 5.45 | 60.9 |
| 6 | C144G | 8.16 | 6.4 | 78.5 |
| 7 | T154N | 6.91 | 5.56 | 80.4 |
| 8 | F191I | 6.41 | 3.32 | 51.8 |
| 9 | V61L | 6.34 | 1.78 | 28 |
| 10 | I80F/M146I | 6.29 | 5.1 | 81.1 |
| 11 | M146I | 3.61 | 3.5 | 96.9 |
| 12 | G85C/A120G/V140I | 2.56 | 2.27 | 88.6 |
| 13 | D237E | 3.84 | 2.94 | 76.5 |
| 14 | F191I/D220E | 3.86 | 2.95 | 76.3 |
| 15 | E109D | 5.23 | 3.89 | 74.5 |
| 16 | A228G | 4.39 | 2.98 | 67.9 |
| 17 | G30S/H131R | 3.16 | 2.02 | 64.1 |
| 18 | N142I/F191L | 4.51 | 1.95 | 43.3 |
| 19 | K82T/P86T/G95A | 25.08 | 2.9 | 11.6 |
| 20 | G95A | 18.23 | 1.28 | 7 |
| 21 | E194K | 3.29 | 0.26 | 7.8 |

(A) High-Active Heat-Resistant Mutant Gn5DH

The enzyme activity and the enzyme activity residual ratio (heat resistance) of the mutant Gn5DH made by the amino acid sequences of sequence numbers 2 to 10 have improved as compared with those of the wild-type (WT) of the sequence number 1.

In the mutant Gn5DH shown in sequence number 2, isoleucine at the 155th position from the N-terminal of the wild-type amino acid sequence in sequence number 1 is substituted with threonine (indicated by abbreviation “I155T”). Similarly, in a mutant Gn5DH shown in sequence number 3, alanine at the 63rd position is substituted with valine, and valine at the 124th position is substituted with isoleucine (“A63V/V124I”). In a mutant Gn5DH shown in sequence number 4, valine at the 254th position is substituted with leucine (“V254L”). In a mutant Gn5DH shown in sequence number 5, threonine at the 154th position is substituted with serine (“T154S”). In a mutant Gn5DH shown in sequence number 6, cysteine at the 144th position is substituted with glycine (“C144G”). In a mutant Gn5DH shown in sequence number 7, threonine at the 154th position is substituted with asparagine acid (“T154N”). In a mutant Gn5DH shown in sequence number 8, phenylalanine at the 191st position is substituted with isoleucine (“F191I”). In a mutant Gn5DH shown in sequence number 9, valine at the 61st position is substituted with leucine (“V61L”). In a mutant Gn5DH shown in sequence number 10, isoleucine at the 80th position is substituted with phenylalanine, and methionine at the 146th position is substituted with isoleucine (“180F/M146I”).

(B) Heat-Resistant Mutant Gn5DH

Moreover, in the mutant Gn5DH made by the amino acid sequences of the sequence numbers 11 to 18, the enzyme activity residual ratio (heat resistance) was improved as compared with that of the wild type (WT).

In a mutant Gn5DH shown in sequence number 11, methionine at the 146th position from the N-terminal of the wild-type amino acid sequence of the sequence number 1 is substituted with isoleucine (“M146I”). Similarly, in a mutant Gn5DH shown in sequence number 12, glycine at the 85th position is substituted with cysteine, and alanine at the 120th position is substituted with glycine, and valine at the 140th position is substituted with isoleucine (“G85C/A120G/V140I”). In a mutant Gn5DH shown in sequence number 13, asparagine acid at the 237th position is substituted with glutamic acid (“D237E”). In a mutant Gn5DH shown in sequence number 14, phenylalanine at the 191st position is substituted with isoleucine and asparagine acid at the 220th position is substituted with glutamic acid (“F191I/D220E”). In a mutant Gn5DH shown in sequence number 15, glutamic acid at the 109th position is substituted with asparagine acid (“E109D”). In a mutant Gn5DH shown in sequence number 16, alanine at the 228th position is substituted with glycine (“A228G”). In a mutant Gn5DH shown in sequence number 17, glycine at the 30th position is substituted with serine, and histidine at the 131st position is substituted with arginine (“G30S/H131R”). In a mutant Gn5DH shown in sequence number 18, asparagine acid at the 142nd position is substituted with isoleucine, and phenylalanine at the 191st position is substituted with leucine (“N142I/F191L”).

(C) High-Active Mutant Gn5DH

Further, in the mutant Gn5DH of the sequence number 19 or 20, the enzyme activity is improved as compared with that of the wild type (WT).

In a mutant Gn5DH shown in sequence number 19, lycine at the 82nd position from the N-terminal of the wild-type amino acid sequence of the sequence number 1 is substituted with threonine, proline at the 86th position is substituted with threonine, and glycine at the 95th position is substituted with alanine (“K82T/P86T/G95A”). Similarly, in a mutant Gn5DH shown in sequence number 20, glycine at the 95th position is substituted with alanine (“G95A”).

### Example 3

### 3. Generation of Mutant Library by Random Mutation of Gn5DH Gene and Screening of High-Active High-Heat-Resistive Mutant (Second Generation)

In the example 2, using the mutant Gn5DH genes of the sequence numbers 2 to 6, 11, 19, and 20 and expressing excellent enzyme activity and high enzyme activity residual ratio as template DNAs, the mutant gene library was generated again by the Error-prone PCR, a mutant library was prepared, and screening was performed by heat treatment at temperature of 58° C. for 45 minutes, and a mutant which passed through the screening was used as a second-generation mutant. In a manner similar to the example 2, second-generation Gn5DH mutants were purified, and an enzyme activity evaluation test and a heat resistance test were conducted. Table 3 illustrates Gn5DH mutants displaying higher activity and higher heat resistance than wild-type Gn5DH as a result of the enzyme activity evaluation test and the heat resistance test (treatment at 53° C. for 10 minutes).

TABLE 3

| | | | | Enzyme |
|---|---|---|---|---|
| | | | Residual | activity |
| | | Enzyme | enzyme | residual |
| Sequence number | | activity | activity | ratio |
| 1 | WT | 6.22 | 0.00 | 0.00 |
| 22 | G95A/M146I | 23.14 | 9.39 | 40.60 |
| 23 | H69R/K82T/P86T/G95A/M146I | 22.73 | 19.38 | 94.77 |
| 24 | A63V/K82T/P86T/G95A/T154S/D237E | 19.92 | 10.67 | 53.56 |
| 25 | K82T/P86T/G95A/M146I/V200A/V254L | 19.37 | 15.09 | 77.89 |
| 26 | K82T/P86T/G95A/M146I | 19.00 | 17.22 | 90.63 |
| 27 | E51K/G95A/M146I | 18.20 | 6.77 | 37.18 |
| 28 | T154S/V254L | 15.05 | 7.02 | 46.65 |
| 29 | T154S/D237E/V254L | 14.20 | 11.35 | 79.92 |
| 30 | D83E/G95A/V200A/V254L | 13.54 | 0.03 | 0.19 |
| 31 | C144G/A227T/V254L | 13.33 | 6.20 | 46.47 |
| 32 | C144G/V254L | 13.04 | 9.49 | 72.77 |
| 33 | F23L/C144G/E201D/V254L | 11.91 | 8.80 | 73.84 |
| 34 | F5Y/E51K/C144G/V254L | 11.86 | 4.14 | 34.90 |
| 35 | I80F/M146I/V254L | 11.52 | 5.82 | 50.48 |
| 36 | A63V/V124I/Q222H | 11.42 | 6.67 | 58.42 |
| 37 | K136M/C144G/E175K/V254L | 11.08 | 7.68 | 69.35 |
| 38 | H131R/C144G/S148G/V254L | 10.86 | 10.51 | 96.74 |
| 39 | A63V/R99C | 10.41 | 6.76 | 64.91 |
| 40 | L24I/I80F/M146I | 9.48 | 6.17 | 65.12 |
| 41 | H54R/A63V/E201V/V254L | 8.62 | 1.18 | 13.71 |
| 42 | H54L/H100Y/C144G/V254L | 8.39 | 4.88 | 58.13 |
| 43 | Q59E/K82T/P86T/G95A/T154S/V254L | 7.48 | 6.98 | 93.37 |
| 44 | E72V/I155T/V254L | 7.43 | 6.49 | 87.32 |
| 45 | A63V/I155T | 7.32 | 2.89 | 39.56 |
| 46 | K82T/P86T/G95A/I155T/V254L | 7.27 | 6.90 | 94.98 |
| 47 | F5Y/E51K/I155T/V254L | 6.97 | 6.13 | 88.03 |
| 48 | E51K/I155T/V254L | 6.95 | 6.51 | 93.64 |
| 49 | I155T/E194G | 6.79 | 2.77 | 40.89 |
| 50 | A63V/V124I/M146I | 6.64 | 5.05 | 76.10 |
| 51 | D3H/G57D/M146I/V254L | 5.79 | 5.10 | 88.16 |
| 52 | G95A/I155T/V254L | 5.35 | 4.12 | 77.02 |
| 53 | I155T/F230L | 5.35 | 4.12 | 77.02 |
| 54 | I155T/I225T/V254L | 4.29 | 3.49 | 81.38 |
| 55 | T103I/M146I | 4.21 | 3.25 | 77.26 |
| 56 | G28D/H69A/G95A/E194G/V254L | 4.12 | 2.45 | 59.40 |
| 57 | L24I/E47K/E51K/I155T/Y190F/E203K | 3.72 | 1.89 | 50.76 |
| 58 | I58F/C144G/E194G | 3.63 | 3.58 | 98.38 |
| 59 | F65Y/M146I/I155T/V254L | 3.42 | 3.19 | 93.15 |
| 60 | A63V/M146I | 3.40 | 2.08 | 61.05 |
| 61 | G9R/E56G/A63V/M146I/D237N | 3.34 | 2.95 | 88.28 |
| 62 | V61I/H69R/M146I | 3.31 | 1.30 | 39.26 |
| 63 | I80F/M146I/I155T | 2.68 | 2.58 | 96.28 |
| 64 | G95A/C144G/V254L | 2.37 | 2.03 | 85.86 |
| 65 | A63T/I155T | 1.02 | 0.14 | 13.71 |
| 66 | A63V/V124I/Q147H | 0.31 | 0.18 | 57.53 |

(D) High-Active Super Heat-Resistant Mutant Gn5DH

The residual enzyme activity of the wild-type (WT) Gn5DH of sequence number 1 after the heat treatment at 53° C. for ten minutes was zero. In contrast, mutant Gn5DH made by the amino acid sequences of sequence numbers 22 to 50 display the enzyme activity also after the heat treatment and display the enzyme activity higher than that of the wild-type Gn5DH also before the heat treatment. Those mutant Gn5DH have higher heat resistance (super heat resistance) than that of the first-generation Gn5DH mutants as Templates.

In the mutant Gn5DH shown in sequence number 22, glycine at the 95th position from the N-terminal of the wild-type amino acid sequence in sequence number 1 is substituted with alanine, and methionine at the 146th position is substituted with isoleucine (“G95A/M146I”). Similarly, in a mutant Gn5DH shown in sequence number 23, histidine at the 69th position is substituted with arginine, lycine at the 82nd position is substituted with threonine, proline at the 86th position is substituted with threonine, glycine at the 95th position is substituted with alanine, and methionine at the 146th position is substituted with isoleucine (“H69R/K82T/P86T/G95A/M146I”). In a mutant Gn5DH shown in sequence number 24, alanine at the 63rd position is substituted with valine, lycine at the 82nd position is substituted with threonine, proline at the 86th position is substituted with threonine, glycine at the 95th position is substituted with alanine, threonine at the 154th position is substituted with serine, and asparagine acid at the 237th position is substituted with glutamic acid (“A63V/K82T/P86T/G95A/T154S/D237E”). In a mutant Gn5DH shown in sequence number 25, lycine at the 82nd position is substituted with threonine, proline at the 86th position is substituted with threonine, glycine at the 95th position is substituted with alanine, methionine at the 146th position is substituted with isoleucine, valine at the 200th position is substituted with alanine, and valine at the 254th position is substituted with leucine (“K82T/P86T/G95A/M1461N200A/V254L”). In a mutant Gn5DH shown in sequence number 26, lycine at the 82nd position is substituted with threonine, proline at the 86th position is substituted with threonine, glycine at the 95th position is substituted with alanine, and methionine at the 146th position is substituted with isoleucine (“K82T/P86T/G95A/M146I”). In a mutant Gn5DH shown in sequence number 27, glutamic acid at the 51st position is substituted with lycine, glycine at the 95th position is substituted with alanine, and methionine at the 146th position is substituted with isoleucine (“E51K/G95A/M146I”). In a mutant Gn5DH shown in sequence number 28, threonine at the 154th position is substituted with serine and valine at the 254th position is substituted with leucine (“T154S/V254L”). In a mutant Gn5DH shown in sequence number 29, threonine at the 154th position is substituted with serine, asparagine acid at the 237th position is substituted with glutamic acid, and valine at the 254th position is substituted with leucine (“T154S/D237E/V254L”). In a mutant Gn5DH shown in sequence number 30, asparagine acid at the 83rd position is substituted with glutamic acid, glycine at the 95th position is substituted with alanine, valine at the 200^{th} position is substituted with alanine, and valine at the 254th position is substituted with leucine (“D83E/G95A/V200A/V254L”). In a mutant Gn5DH shown in sequence number 31, cysteine at the 144th position is substituted with glycine, alanine at the 227th position is substituted with threonine, and valine at the 254th position is substituted with leucine (“C144G/A227T/V254L”). In a mutant Gn5DH shown in sequence number 32, cysteine at the 144th position is substituted with glycine, and valine at the 254th position is substituted with leucine (“C144G/V254L”).

Moreover, in a mutant Gn5DH shown in sequence number 33, phenylalanine at the 23rd position is substituted with leucine, cysteine at the 144th position is substituted with glycine, glutamic acid at the 201st position is substituted with asparagine acid, and valine at the 254th position is substituted with leucine (“F23L/C144G/E201D/V254L”). In a mutant Gn5DH shown in sequence number 34, phenylalanine at the fifth position is substituted with tyrosine, glutamic acid at the 51st position is substituted with lycine, cysteine at the 144th position is substituted with glycine, and valine at the 254th position is substituted with leucine (“F5Y/E51K/C144G/V254L”). In a mutant Gn5DH shown in sequence number 35, isoleucine at the 80th position is substituted with phenylalanine, methionine at the 146th position is substituted with isoleucine, and valine at the 254th position is substituted with leucine (I180F/M146I/V254L″). In a mutant Gn5DH shown in sequence number 36, alanine at the 63rd position is substituted with valine, valine at the 124th position is substituted with isoleucine, and glutamine at the 222nd position is substituted with histidine (“A63V/V124I/Q222H”). In a mutant Gn5DH shown in sequence number 37, lycine at the 136th position is substituted with methionine, cysteine at the 144th position is substituted with glycine, glutamic acid at the 175th position is substituted with lycine, and valine at the 254th position is substituted with leucine (“K136M/C144G/E175K/V254L”). In a mutant Gn5DH shown in sequence number 38, histidine at the 131st position is substituted with arginine, cysteine at the 144th position is substituted with glycine, and valine at the 254th position is substituted with leucine (“H131R/C144G/S148G/V254L”). In a mutant Gn5DH shown in sequence number 39, alanine at the 63rd position is substituted with valine, and arginine at the 99th position is substituted with cysteine (“A63V/R99C”). In a mutant Gn5DH shown in sequence number 40, leucine at the 24th position is substituted with isoleucine, isoleucine at the 80th position is substituted with phynylalanine, and methionine at the 146th position is substituted with isoleucine (“L24I/I80F/M146I”). In a mutant Gn5DH shown in sequence number 41, histidine at the 54th position is substituted with arginine, alanine at the 63rd position is substituted with valine, glutamic acid at the 201st position is substituted with asparagine acid, and valine at the 254th position is substituted with leucine (“H54R/A63V/E201V/V254L”). In a mutant Gn5DH shown in sequence number 42, histidine at the 54th position is substituted with leucine, histidine at the 100th position is substituted with tyrosine, serine at the 144th position is substituted with glycine, and valine at the 254th position is substituted with leucine (“H54L/H100Y/C144G/V254L”).

Further, in a mutant Gn5DH shown in sequence number 43, glutamine at the 59th position is substituted with glutamic acid, lycine at the 82nd position is substituted with threonine, proline at the 86th position is substituted with threonine, glycine at the 95th position is substituted with alanine, threonine at the 154th position is substituted with serine, and valine at the 254th position is substituted with leucine (“Q59E/K82T/P86T/G95A/T154S/V254L”). In a mutant Gn5DH shown in sequence number 44, glutamic acid at the 72nd position is substituted with valine, isoleucine at the 155th position is substituted with threonine, and valine at the 254th position is substituted with leucine (“E72V/1155T”). In a mutant Gn5DH shown in sequence number 45, alanine at the 63rd position is substituted with valine, and isoleucine at the 155th position is substituted with threonine (“A63V/I155T”). In a mutant Gn5DH shown in sequence number 46, lycine at the 82nd position is substituted with threonine, proline at the 86th position is substituted with threonine, glycine at the 95th position is substituted with alanine, isoleucine at the 155th position is substituted with threonine, and valine at the 254th position is substituted with leucine (“K82T/P86T/G95A/1155T/V254L”). In a mutant Gn5DH shown in sequence number 47, phenylalanine at the 5th position is substituted with tyrosine, glutamic acid at the 51st position is substituted with lycine, isoleucine at the 155th position is substituted with threonine, and valine at the 254th position is substituted with leucine (“F5Y/E51K/1155T/V254L”). In a mutant Gn5DH shown in sequence number 48, glutamic acid at the 51st position is substituted with lycine, isoleucine at the 155th position is substituted with threonine, and valine at the 254th position is substituted with leucine (“E51K/I155T/V254L”). In a mutant Gn5DH shown in sequence number 49, isoleucine at the 155th position is substituted with threonine, and glutamic acid at the 194th position is substituted with glycine (I“155T/E194G”). In a mutant Gn5DH shown in sequence number 50, alanine at the 63rd position is substituted with valine, valine at the 124th position is substituted with isoleucine, and methionine at the 146th position is substituted with isoleucine (“A63V/V124I/M146I”).

(E) Super Heat-Resistant Mutant Gn5DH

Moreover, mutants Gn5DH made by amino acid sequences of sequence numbers 51 to 66 displayed enzyme activity also after heat treatment, and have improved enzyme activity residual ratio (heat resistance) as compared with that of mutants of the wild type (WT). The mutants Gn5DH have higher heat resistance (super heat resistance) than that of first-generation Gn5DH mutants as Templates.

In a mutant Gn5DH shown in sequence number 51, asparagine acid at the third position from the N-terminal of the wild-type amino acid sequence in sequence number 1 is substituted with histidine, glycine at the 57th position is substituted with asparagine acid, methionine at the 146th position is substituted with isoleucine, and valine at the 254th position is substituted with leucine (“D3H/G57D/M146I/V254L”). Similarly, in a mutant Gn5DH shown in sequence number 52, glycine at the 95th position is substituted with alanine, isoleucine at the 155th position is substituted with threonine, and valine at the 254th position is substituted with leucine (“G95A/I155T/V254L”). In a mutant Gn5DH shown in sequence number 53, isoleucine at the 155th position is substituted with threonine and phenylalanine at the 230th position is substituted with leucine (“1155T/F230L”). In a mutant Gn5DH shown in sequence number 54, isoleucine at the 155th position is substituted with threonine, isoleucine at the 225th position is substituted with threonine, and valine at the 254th position is substituted with leucine (I“155T/I225T/V254L”). In a mutant Gn5DH shown in sequence number 55, threonine at the 103rd position is substituted with isoleucine, and methionine at the 146th position is substituted with isoleucine (“T103I/M146I”). In a mutant Gn5DH shown in sequence number 56, glycine at the 28th position is substituted with asparagine acid, histidine at the 69th position is substituted with alanine, glycine at the 95th position is substituted with alanine, glutamic acid at the 194th position is substituted with glutamine, and valine at the 254th position is substituted with leucine (“G28D/H69A/G95A/E194G/V254L”). In a mutant Gn5DH shown in sequence number 57, lycine at the 24th position is substituted with isoleucine, glutamic acid at the 47th position is substituted with lycine, glutamic acid at the 51st position is substituted with lycine, isoleucine at the 155th position is substituted with threonine, tyrosine at the 190th position is substituted with phenylalanine, and glutamic acid at the 203rd position is substituted with lycine (“L24I/E47K/E51K/I155T/Y190F/E203K”). In a mutant Gn5DH shown in sequence number 58, isoleucine at the 58th position is substituted with phenylalanine, cysteine at the 144th position is substituted with glycine, and glutamic acid at the 194th position is substituted with glycine (“I58F/C144G/E194G”).

Moreover, in a mutant Gn5DH shown in sequence number 59, phenylalanine at the 65th position is substituted with tyrosine, methionine at the 146th position is substituted with isoleucine, isoleucine at the 155th position is substituted with threonine, and valine at the 254th position is substituted with leucine (“F65Y/M146I/1155T/V254V”). In a mutant Gn5DH shown in sequence number 60, alanine at the 63rd position is substituted with valine, and methionine at the 146th position is substituted with isoleucine (“A63V/M146I”). In a mutant Gn5DH shown in sequence number 61, glycine at the ninth position is substituted with arginine, glutamic acid at the 56th position is substituted with glycine, alanine at the 63rd position is substituted with valine, methionine at the 146th position is substituted with isoleucine, and asparagine acid at the 237th position is substituted with asparagine (“G9R/E56G/A63V/M146I/D237N”). In a mutant Gn5DH shown in sequence number 62, valine at the 61st position is substituted with isoleucine, histidine at the 69th position is substituted with arginine, and methionine at the 146th position is substituted with isoleucine (“V61I/H69R/M146I”). In a mutant Gn5DH shown in sequence number 63, isoleucine at the 80th position is substituted with phenylalanine, methionine at the 146th position is substituted with isoleucine, and isoleucine at the 155th position is substituted with leucine (“I80F/M146I/I155L”). In a mutant Gn5DH shown in sequence number 64, glycine at the 95th position is substituted with alanine, cysteine at the 144th position is substituted with glycine, and valine at the 254th position is substituted with leucine (“G95A/C144G/V254L”). In a mutant Gn5DH shown in sequence number 65, alanine at the 63rd position is substituted with threonine, and isoleucine at the 155th position is substituted with threonine (“A63T/I155T). In a mutant Gn5DH shown in sequence number 66, alanine at the 63rd position is substituted with valine, valine at the 124th position is substituted with isoleucine, and glutamine at the 147th position is substituted with histidine (“A63V/V124I/Q147H”).

### Example 4

### 3. Generation of Mutant Library by Random Mutation of Gn5DH Gene and Screening of High-Active High-Heat-Resistive Mutant (Third Generation)

In the example 3, using mutant Gn5DH genes expressing excellent enzyme activity and high enzyme activity residual ratio as Template DNAs, the mutant gene library was generated again by the Error-prone PCR, the mutant library was prepared, and screening was performed on third-generation mutants. In a manner similar to the example 3, third-generation Gn5DH mutants were purified, and an enzyme activity evaluation test and a heat resistance test were conducted.

Table 4 shows Gn5DH mutants displaying higher activity and higher heat resistance that wild-type Gn5DH as a result of the enzyme activity evaluation test and the heat resistance test (treatment at 57.5° C. for 10 minutes).

TABLE 4

| | | | | Enzyme |
|---|---|---|---|---|
| | | | Residual | activity |
| | | Enzyme | enzyme | residual |
| Sequence number | | activity | activity | ratio |
| 1 | WT | 7.6 | 0.0 | 0 |
| 70 | H69R K82T P86T G95A E109D | 30.9 | 27.1 | 88 |
| | M146I | | | |
| 71 | H69R K82T P86T Q95A M146I V254L | 20.5 | 16.4 | 80 |
| 72 | A63V K82T P86T G95A H131R | 12.5 | 12.2 | 98 |
| | C144G S148C V254L | | | |
| 73 | K82T P86T H131R C144G S148C | 11.9 | 11.4 | 96 |
| | E203V V254L | | | |
| 74 | I112V T42L E44K K82T P86T G95A | 11.1 | 10.0 | 89 |
| | E134G I155T V254L | | | |
| 75 | K82T P86T H131R C144G S148C | 10.8 | 10.4 | 96 |
| | V254L | | | |
| 76 | K82T P86T G95A I155T G163R F191I | 10.4 | 8.8 | 85 |
| | V254L | | | |
| 77 | A63V K82T P86T G95A H131R | 9.0 | 8.3 | 93 |
| | C144G S148C K234R G248L V254L | | | |
| 78 | H69R K82T P86T G95A I155T M250L | 8.7 | 8.7 | 100 |
| | V254L | | | |
| 79 | L25M I155T K234T V254L | 8.5 | 7.9 | 92 |
| 80 | Q35 H69R K82T P86T G95A A120G | 8.3 | 8.5 | 102 |
| | M146I F191L V254L | | | |
| 81 | P86H H131R C144G S148C V254L | 8.2 | 7.6 | 93 |
| 82 | A63V K82T P86T G95A D112E I155T | 8.2 | 7.6 | 92 |
| | V254L | | | |
| 83 | H69R K82T P86T H131R C144G | 8.1 | 7.7 | 96 |
| | S148C V254L | | | |
| 84 | K82T P86T G95A E109D I155T V254L | 8.0 | 7.7 | 97 |
| 85 | I155T V254L | 8.0 | 7.0 | 88 |
| 86 | H69R K82T I84F P86T G95A I155T | 7.8 | 7.8 | 100 |
| | V254L | | | |
| 87 | H69R K82T P86T H131R C144G | 7.2 | 6.7 | 93 |
| | S148C V200A V254L | | | |
| 88 | A63V K82T P86T G95A I155T D237E | 7.1 | 6.4 | 90 |
| | V254L | | | |
| 89 | A63V H131R C144G S148C V254L | 7.0 | 6.6 | 95 |
| 90 | E56D K82T P86T G95A I155T V254L | 6.9 | 5.7 | 83 |
| 91 | Q59E A63V K82T P86T G95A I155T | 6.6 | 5.8 | 87 |
| | E203D V254L | | | |
| 92 | H69R K82T P86T G95A I155T V254L | 6.2 | 5.8 | 93 |
| 93 | H69R K82T P86T G95A R99L I155T | 6.2 | 5.8 | 94 |
| | V254L | | | |
| 94 | L48P F122S H131R C144G S148C | 6.1 | 5.7 | 94 |
| | V254L | | | |
| 95 | T42A E78D K82T P86T G95A I155T | 6.0 | 2.0 | 34 |
| | F230Y V254L | | | |
| 96 | Q59E K82T P86T G95A M146I I155T | 5.8 | 5.7 | 97 |
| | G248L V254L | | | |
| 97 | Q59E K82T P86T G95A A137T M146I | 5.8 | 5.6 | 96 |
| | I155T F230S V254L | | | |
| 98 | K82T P86T G95A M146I I155T V254L | 5.5 | 5.3 | 97 |
| 99 | K82T P86T G95A I155T D237E V254L | 5.2 | 4.9 | 93 |
| 100 | E51D A63V D83N N93T H100F | 4.9 | 4.7 | 97 |
| | D112E H131R C144G S148C A228G | | | |
| | V254L | | | |
| 101 | H69R K82T P86T G95A V128I H131R | 4.5 | 4.3 | 95 |
| | C144G S148C K192I V254L | | | |
| 102 | K82T P86T G95A I155T V200A V254L | 4.3 | 3.6 | 85 |
| 103 | Q59E C144G S148C I155T G248L | 3.7 | 3.4 | 92 |
| | V254L | | | |
| 104 | G95S I155T V254L | 2.4 | 2.3 | 92 |
| 105 | H69R K82T P86T G95A M146I I155L | 0.8 | 0.8 | 98 |
| 106 | G95A M146I I155L V254L | 0.1 | 0.1 | 99 |

(F) High-Active Extra-Super Heat-Resistant Mutant Gn5DH

The residual enzyme activity of the wild-type (WT) Gn5DH of sequence number 1 after the heat treatment at 57.5° C. for ten minutes was zero. In contrast, mutant Gn5DH made by the amino acid sequences of sequence numbers 70 to 76 display the enzyme activity also after the heat treatment and display the enzyme activity higher than that of the wild-type Gn5DH also before the heat treatment. Those mutant Gn5DH have higher heat resistance (extra-super heat resistance) than that of the second-generation Gn5DH mutants as Templates.

(G) Extra-Super Heat-Resistant Mutant Gn5DH

Moreover, the mutant Gn5DH made by the amino acid sequences of sequence numbers 77 to 106 display the enzyme activity also after the heat treatment, and display the enzyme activity residual ratio higher than that of the wild-type (WT) Gn5DH. Those mutant Gn5DH have higher heat resistance (extra-super heat resistance) than that of the second-generation Gn5DH mutants as templates.

### INDUSTRIAL APPLICABILITY

The mutant-type proteins having gluconate dehydrogenase activity according to the present disclosure can be used for techniques such as production of useful materials, production, measurement, or analysis of energy-related materials, environment preservation, medical services, and the like. Use of the mutant-type proteins to an electrochemical device, particularly, a biosensor, and an enzyme battery is expected.

1-16. (canceled)

16. A mutant gluconate dehydrogenase made by an amino acid sequence obtained by deleting, substituting, adding or inserting one or plural amino acids in an amino acid sequence represented by sequence number 1,
the mutant gluconate dehydrogenase displaying enzyme activity which is equal to or higher than 120% of that of a wild-type gluconate dehydrogenase made by an amino acid sequence represented by sequence number 1.

17. The mutant gluconate dehydrogenase of claim 16, wherein residual enzyme activity after heat treatment performed at 47.5° C. for 10 minutes is equal to or higher than 20% of the enzyme activity before the heat treatment.

18. The mutant gluconate dehydrogenase of claim 17, wherein residual enzyme activity after heat treatment performed at 53° C. for 10 minutes is equal to or higher than 20% of the enzyme activity before the heat treatment.

19. The mutant gluconate dehydrogenase of claim 18, wherein residual enzyme activity after heat treatment performed at 57.5° C. for 10 minutes is equal to or higher than 20% of the enzyme activity before the heat treatment.

20. The mutant gluconate dehydrogenase of claim 19, which is represented by any of sequence numbers 70 to 76.

21. The mutant gluconate dehydrogenase of claim 18, which is represented by any of sequence numbers 22 to 29, 31 to 40, and 42.

22. The mutant gluconate dehydrogenase of claim 17, which is represented by any of sequence numbers 2 to 6.

23. A mutant gluconate dehydrogenase made by an amino acid sequence obtained by deleting, substituting, adding or inserting one or plural amino acids in an amino acid sequence represented by sequence number 1,
wherein residual enzyme activity after heat treatment performed at 47.5° C. for 10 minutes is equal to or higher than 20% of enzyme activity before the heat treatment.

24. The mutant gluconate dehydrogenase of claim 23, wherein residual enzyme activity after heat treatment performed at 53° C. for 10 minutes is equal to or higher than 20% of enzyme activity before the heat treatment.

25. The mutant gluconate dehydrogenase of claim 24, wherein residual enzyme activity after heat treatment performed at 57.5° C. for 10 minutes is equal to or higher than 20% of the enzyme activity before the heat treatment.

26. The mutant gluconate dehydrogenase of claim 25, which is represented by any of sequence numbers 77 to 106.

27. The mutant gluconate dehydrogenase of claim 24, which is represented by any of sequence numbers 43 to 64, and 66.

28. The mutant gluconate dehydrogenase of claim 23, which is represented by any of sequence numbers 7 to 18.

29. The mutant gluconate dehydrogenase of claim 16, which is represented by sequence number 19 or 20.

30. An electrochemical device using enzyme, characterized in that the enzyme is a mutant gluconate dehydrogenase made by an amino acid sequence obtained by deleting, substituting, adding or inserting one or plural amino acids in an amino acid sequence represented by sequence number 1, and
the mutant gluconate dehydrogenase displays enzyme activity which is equal to or higher than 120% of that of a wild-type gluconate dehydrogenase made by an amino acid sequence represented by sequence number 1.


SEQUENCE LISTING (106 sequences)

SEQ ID NO: 1 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 2 - protein, 254 aa, Artificial Sequence
  Synthesized I155T mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 3 - protein, 254 aa, Artificial Sequence
  Synthesized A63V/V124I mutant
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRRHPFTEFPEQEWNDVIAVNQTAVFLISQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 4 - protein, 254 aa, Artificial Sequence
  Synthesized V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 5 - protein, 254 aa, Artificial Sequence
  Synthesized T154S mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDSITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 6 - protein, 254 aa, Artificial Sequence
  Synthesized C144G mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINIGSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 7 - protein, 254 aa, Artificial Sequence
  Synthesized T154N mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDNITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 8 - protein, 254 aa, Artificial Sequence
  Synthesized F191I mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYIKTEMTKALVEDEAFTAWLCKRTPAARWGDPQELIGAAVFLSSKASDFVNGHLLFVDGGMLVAV

SEQ ID NO: 9 - protein, 254 aa, Artificial Sequence
  Synthesized V61L mutant
MNDLFSLAGKNILITGSAQGIGFLLATGLGKYGAQIIINDITAERAELAVEKLHQEGIQALAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 10 - protein, 254 aa, Artificial Sequence
  Synthesized I80F/M146I mutant
MNDLFSLAGKNILITGSAQGIGFLLATGLGKYGAQIIINDITAERAELAVEKLHQEGIQAVAAPFNVTHKHEIDAAVEHFEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 11 - protein, 254 aa, Artificial Sequence
  Synthesized M146I mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 12 - protein, 254 aa, Artificial Sequence
  Synthesized G85C/A120G/V140I mutant
MNDLFSLAGKNILITGSAQGIGFLLATGLGKYGAQIIINDITAERAELAVEKLHQEGIQAVAAPFNVTHKHEIDAAVEHIEKDICPIDVLVNNAGIQRRHPFTEFPEQEWNDVIAVNQTGVFLVSQAVTRHMVERKAGKIINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 13 - protein, 254 aa, Artificial Sequence
  Synthesized D237E mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASEFVNGHLLFVDGGMLVAV

SEQ ID NO: 14 - protein, 254 aa, Artificial Sequence
  Synthesized F191I/D220E mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYIKTEMTKALVEDEAFTAWLCKRTPAARWGEPQELIGAAVFLSSKASDFVNGHLLFVDGGMLVAV

SEQ ID NO: 15 - protein, 254 aa, Artificial Sequence
  Synthesized E109D mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQDWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 16 - protein, 254 aa, Artificial Sequence
  Synthesized A228G mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAGVFLSSKASDFVNGHLLFVDGGMLVAV

SEQ ID NO: 17 - protein, 254 aa, Artificial Sequence
  Synthesized G30S/H131R mutant
MNDLFSLAGKNILITGSAQGIGFLLATGLSKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRRMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 18 - protein, 254 aa, Artificial Sequence
  Synthesized N142I/F191L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVIIICSMQSELGRDTITPYAASKGAVKMLTRGMCVELARHNIQVNGIAPGYLKTEMTKALVEDEAFTAWLCKRTPAARWGDPQELIGAAVFLSSKASDFVNGHLLFVDGGMLVAV

SEQ ID NO: 19 - protein, 254 aa, Artificial Sequence
  Synthesized K82T/P86T/G95A mutant
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 20 - protein, 254 aa, Artificial Sequence
  Synthesized G95A mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAAIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 21 - protein, 254 aa, Artificial Sequence
  Synthesized E194K mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTKMTKALVEDEAFTAWLCKRTPAARWGDPQELIGAAVFLSSKASDFVNGHLLFVDGGMLVAV

SEQ ID NO: 22 - protein, 254 aa, Artificial Sequence
  Synthesized G95A/M146I mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAAIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 23 - protein, 254 aa, Artificial Sequence
  Synthesized H69R/K82T/P86T/G95A/M146I mutant
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 24 - protein, 254 aa, Artificial Sequence
  Synthesized A63V/K82T/P86T/G95A/T154S/D237E mutant
MNDLFSLAGKNILITGSAQGIGFLLATGLGKYGAQIIINDITAERAELAVEKLHQEGIQAVAVPFNVTHKHEIDAAVEHIETDIGTIDVLVNNAAIQRRHPFTEFPEQEWNDVIAVNQTAVFLVSQAVTRHMVERKAGKVINICSMQSELGRDSITPYAASKGAVKMLTRGMCVELARHNIQVNGIAPGYFKTEMTKALVEDEAFTAWLCKRTPAARWGDPQELIGAAVFLSSKASEFVNGHLLFVDGGMLVAV

SEQ ID NO: 25 - protein, 254 aa, Artificial Sequence
  Synthesized K82T/P86T/G95A/M146I/V200A/V254L mutant
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALAEDEAFTAWLCKRTPAARWGDPQELIGAAVFLSSKASDFVNGHLLFVDGGMLVAL

SEQ ID NO: 26 - protein, 254 aa, Artificial Sequence
  Synthesized K82T/P86T/G95A/M146I mutant
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 27 - protein, 254 aa, Artificial Sequence
  Synthesized E51K/G95A/M146I mutant
MNDLFSLAGKNILITGSAQGIGFLLATGLGKYGAQIIINDITAERAELAVKKLHQEGIQAVAAPFNVTHKHEIDAAVEHIEKDIGPIDVLVNNAAIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 28 - protein, 254 aa, Artificial Sequence
  Synthesized T154S/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDSITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 29 - protein, 254 aa, Artificial Sequence
  Synthesized T154S/D237E/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDSITPYAASKGAVKMLTRGMCVELARHNIQVNGIAPGYFKTEMTKALVEDEAFTAWLCKRTPAARWGDPQELIGAAVFLSSKASEFVNGHLLFVDGGMLVAL

SEQ ID NO: 30 - protein, 254 aa, Artificial Sequence
  Synthesized D83E/G95A/V200A/V254L mutant
MNDLFSLAGKNILITGSAQGIGFLLATGLGKYGAQIIINDITAERAELAVEKLHQEGIQAVAAPFNVTHKHEIDAAVEHIEKEIGPIDVLVNNAAIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALAEDEAFTAWLCKRTPAARWGDPQELIGAAVFLSSKASDFVNGHLLFVDGGMLVAL

SEQ ID NO: 31 - protein, 254 aa, Artificial Sequence
  Synthesized C144G/A227T/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINIGSMQSELGRDTITPYAASKGAVKMLTRGMCVELARHNIQVNGIAPGYFKTEMTKALVEDEAFTAWLCKRTPAARWGDPQELIGATVFLSSKASDFVNGHLLFVDGGMLVAL

SEQ ID NO: 32 - protein, 254 aa, Artificial Sequence
  Synthesized C144G/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINIGSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 33 - protein, 254 aa, Artificial Sequence
  Synthesized F23L/C144G/E201D/V254L mutant
MNDLFSLAGKNILITGSAQGIGLLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINIGSMQSELGRDTITPYAASKGAVKMLTRGMCVELARHNIQVNGIAPGYFKTEMTKALVDDEAFTAWLCKRTPAARWGDPQELIGAAVFLSSKASDFVNGHLLFVDGGMLVAL

SEQ ID NO: 34 - protein, 254 aa, Artificial Sequence
  Synthesized F5Y/E51K/C144G/V254L mutant
MNDLYSLAGKNILITGSAQGIGFLLATGLGKYGAQIIINDITAERAELAVKKLHQEGIQAVAAPFNVTHKHEIDAAVEHIEKDIGPIDVLVNNAGIQRRHPFTEFPEQEWNDVIAVNQTAVFLVSQAVTRHMVERKAGKVINIGSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 35 - protein, 254 aa, Artificial Sequence
  Synthesized I80F/M146I/V254L mutant
MNDLFSLAGKNILITGSAQGIGFLLATGLGKYGAQIIINDITAERAELAVEKLHQEGIQAVAAPFNVTHKHEIDAAVEHFEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 36 - protein, 254 aa, Artificial Sequence
  Synthesized A63V/V241I/Q222H mutant
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRRHPFTEFPEQEWNDVIAVNQTAVFLISQAVTRHMVERKAGKVINICSMQSELGRDTITPYAASKGAVKMLTRGMCVELARHNIQVNGIAPGYFKTEMTKALVEDEAFTAWLCKRTPAARWGDPHELIGAAVFLSSKASDFVNGHLLFVDGGMLVAV

SEQ ID NO: 37 - protein, 254 aa, Artificial Sequence
  Synthesized K136M/C144G/E175K/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERMAGKVINIGSMQSELGRDTITPYAASKGAVKMLTRGMCVKLARHNIQVNGIAPGYFKTEMTKALVEDEAFTAWLCKRTPAARWGDPQELIGAAVFLSSKASDFVNGHLLFVDGGMLVAL

SEQ ID NO: 38 - protein, 254 aa, Artificial Sequence
  Synthesized C131R/C144G/S148G/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRRMVERKAGKVINIGSMQGELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 39 - protein, 254 aa, Artificial Sequence
  Synthesized A63V/R99C mutant
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRCHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 40 - protein, 254 aa, Artificial Sequence
  Synthesized L24I/I80F/M146I mutant
MNDLFSLAGKNILITGSAQGIGFILATGLGKYGAQIIINDITAERAELAVEKLHQEGIQAVAAPFNVTHKHEIDAAVEHFEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 41 - protein, 254 aa, Artificial Sequence
  Synthesized H54R/A63V/E201V/V254L mutant
MNDLFSLAGKNILITGSAQGIGFLLATGLGKYGAQIIINDITAERAELAVEKLRQEGIQAVAVPFNVTHKHEIDAAVEHIEKDIGPIDVLVNNAGIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTEMTKALVVDEAFTAWLCKRTPAARWGDPQELIGAAVFLSSKASDFVNGHLLFVDGGMLVAL

SEQ ID NO: 42 - protein, 254 aa, Artificial Sequence
  Synthesized H54L/H100Y/C144G/V254L mutant
MNDLFSLAGKNILITGSAQGIGFLLATGLGKYGAQIIINDITAERAELAVEKLLQEGIQAVAAPFNVTHKHEIDAAVEHIEKDIGPIDVLVNNAGIQRRYPFTEFPEQEWNDVIAVNQTAVFLVSQAVTRHMVERKAGKVINIGSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 43 - protein, 254 aa, Artificial Sequence
  Synthesized Q59E/K82T/P86T/G95A/T154S/V254L mutant
MNDLFSLAGKNILITGSAQGIGFLLATGLGKYGAQIIINDITAERAELAVEKLHQEGIEAVAAPFNVTHKHEIDAAVEHIETDIGTIDVLVNNAAIQRRHPFTEFPEQEWNDVIAVNQTAVFLVSQAVTRHMVERKAGKVINICSMQSELGRDSITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 44 - protein, 254 aa, Artificial Sequence
  Synthesized E72V/I155T/V254L mutant
MNDLFSLAGKNILITGSAQGIGFLLATGLGKYGAQIIINDITAERAELAVEKLHQEGIQAVAAPFNVTHKHVIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 45 - protein, 254 aa, Artificial Sequence
  Synthesized A63V/I155T mutant
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 46 - protein, 254 aa, Artificial Sequence
  Synthesized K82T/P86T/G95A/I155T/V254L mutant
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 47 - protein, 254 aa, Artificial Sequence
  Synthesized F5Y/E51K/I155T/V254L mutant
MNDLYSLAGKNILITGSAQGIGFLLATGLGKYGAQIIINDITAERAELAVKKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 48 - protein, 254 aa, Artificial Sequence
  Synthesized E51K/I155T/V254L mutant
MNDLFSLAGKNILITGSAQGIGFLLATGLGKYGAQIIINDITAERAELAVKKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 49 - protein, 254 aa, Artificial Sequence
  Synthesized I155T/E194G mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTGMTKALVEDEAFTAWLCKRTPAARWGDPQELIGAAVFLSSKASDFVNGHLLFVDGGMLVAV

SEQ ID NO: 50 - protein, 254 aa, Artificial Sequence
  Synthesized A63V/V124I/M146I mutant
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRRHPFTEFPEQEWNDVIAVNQTAVFLISQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 51 - protein, 254 aa, Artificial Sequence
  Synthesized D3H/G57D/M146I/V254L mutant
MNHLFSLAGKNILITGSAQGIGFLLATGLGKYGAQIIINDITAERAELAVEKLHQEDIQAVAAPFNVTHKHEIDAAVEHIEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 52 - protein, 254 aa, Artificial Sequence
  Synthesized G95A/I155T/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 53 - protein, 254 aa, Artificial Sequence
  Synthesized I155T/F230L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVLLSSKASDFVNGHLLFVDGGMLVAV

SEQ ID NO: 54 - protein, 254 aa, Artificial Sequence
  Synthesized I155T/I225T/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELTGAAVFLSSKASDFVNGHLLFVDGGMLVAL

SEQ ID NO: 55 - protein, 254 aa, Artificial Sequence
  Synthesized T103I/M146I mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFIEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 56 - protein, 254 aa, Artificial Sequence
  Synthesized G28D/H69A/G95A/E194G/V254L mutant
MNDLFSLAGKNILITGSAQGIGFLLATDLGKYGAQIIINDITAERAELAVEKLHQEGIQAVAAPFNVTAKHEIDAAVEHIEKDIGPIDVLVNNAAIQRRHPFTEFPEQEWNDVIAVNQTAVFLVSQAVTRHMVERKAGKVINICSMQSELGRDTITPYAASKGAVKMLTRGMCVELARHNIQVNGIAPGYFKTGMTKALVEDEAFTAWLCKRTPAARWGDPQELIGAAVFLSSKASDFVNGHLLFVDGGMLVAL

SEQ ID NO: 57 - protein, 254 aa, Artificial Sequence
  Synthesized L24I/E47K/E51K/I155T/Y190F/E203K mutant
MNDLFSLAGKNILITGSAQGIGFILATGLGKYGAQIIINDITAERAKLAVKKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGFFKTEMTKALVEDKAFTAWLCKRTPAARWGDPQELIGAAVFLSSKASDFVNGHLLFVDGGMLVAV

SEQ ID NO: 58 - protein, 254 aa, Artificial Sequence
  Synthesized I58F/C144G/E194G mutant
MNDLFSLAGKNILITGSAQGIGFLLATGLGKYGAQIIINDITAERAELAVEKLHQEGFQAVAAPFNVTHKHEIDAAVEHIEKDIGPIDVLVNNAGIQRRHPFTEFPEQEWNDVIAVNQTAVFLVSQAVTRHMVERKAGKVINIGSMQSELGRDTITPYAASKGAVKMLTRGMCVELARHNIQVNGIAPGYFKTGMTKALVEDEAFTAWLCKRTPAARWGDPQELIGAAVFLSSKASDFVNGHLLFVDGGMLVAV

SEQ ID NO: 59 - protein, 254 aa, Artificial Sequence
  Synthesized F65Y/M146I/I155T/V254L mutant
MNDLFSLAGKNILITGSAQGIGFLLATGLGKYGAQIIINDITAERAELAVEKLHQEGIQAVAAPYNVTHKHEIDAAVEHIEKDIGPIDVLVNNAGIQRRHPFTEFPEQEWNDVIAVNQTAVFLVSQAVTRHMVERKAGKVINICSIQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 60 - protein, 254 aa, Artificial Sequence
  Synthesized A63V/M146I mutant
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 61 - protein, 254 aa, Artificial Sequence
  Synthesized G9R/E56G/A63V/M146I/D237N mutant
MNDLFSLARKNILITGSAQGIGFLLATGLGKYGAQIIINDITAERAELAVEKLHQGGIQAVAVPFNVTHKHEIDAAVEHIEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASNFVNGHLLFVDGGMLVAV

SEQ ID NO: 62 - protein, 254 aa, Artificial Sequence
  Synthesized V61I/H69R/M146I mutant
MNDLFSLAGKNILITGSAQGIGFLLATGLGKYGAQIIINDITAERAELAVEKLHQEGIQAIAAPFNVTHKHEIDAAVERIEKDIGPIDVLVNNAGIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 63 - protein, 254 aa, Artificial Sequence
  Synthesized I80F/M146I/I155T mutant
MNDLFSLAGKNILITGSAQGIGFLLATGLGKYGAQIIINDITAERAELAVEKLHQEGIQAVAAPFNVTHKHEIDAAVEHFEKDIGPIDVLVNNAGIQRRHPFTEFPEQEWNDVIAVNQTAVFLVSQAVTRHMVERKAGKVINICSIQSELGRDTLTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 64 - protein, 254 aa, Artificial Sequence
  Synthesized G95A/C144G/V254L mutant
MNDLFSLAGKNILITGSAQGIGFLLATGLGKYGAQIIINDITAERAELAVEKLHQEGIQAVAAPFNVTHKHEIDAAVEHIEKDIGPIDVLVNNAAIQRRHPFTEFPEQEWNDVIAVNQTAVFLVSQAVTRHMVERKAGKVINIGSMQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 65 - protein, 254 aa, Artificial Sequence
  Synthesized A63T/I155T mutant
MNDLFSLAGKNILITGSAQGIGFLLATGLGKYGAQIIINDITAERAELAVEKLHQEGIQAVATPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 66 - protein, 254 aa, Artificial Sequence
  Synthesized A63V/V124I/Q147H mutant
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRRHPFTEFPEQEWNDVIAVNQTAVFLISQAVTRHMVERKAGKVINICSMHSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 67 - DNA, 765 bp, Escherichia coli
atgaacgatctattttcactggcaggaaaaaatatcttgattaccggttcagcacagggcattggctttttactggcaaccggcctgggtaaatatggcgcacaaataattattaatgatattactgccgaacgcgcagaacttgctgtagaaaaactccaccaggagggtattcaggccgttgccgcaccttttaatgttactcataaacatgaaattgatgccgccgttgaacatatcgaaaaggacatcggccccattgatgtgctggtgaataacgccggtatccagcgccgtcatccttttactgagttccctgaacaagagtggaatgatgtgatcgcagtaaaccagaccgccgtgttcctggtatcgcaagcggtaactcgtcacatggttgaacgcaaggcaggtaaagttattaatatttgctcgatgcaaagcgaactgggacgtgacaccatcaccccttatgccgcatcgaaaggggcggtaaaaatgctcacccgcggcatgtgcgtcgagctggcgcgccacaatattcaggtcaacggtattgcgccgggctatttcaaaacagaaatgactaaagcactggttgaggacgaagccttcaccgcctggttgtgcaaacggacccccgccgcacgctggggagatccgcaggaactgattggtgctgcggtgttcctttcttcaaaagcctctgatttcgtaaacggccacctgttgtttgttgatggcggcatgttagtggctgtttaa

SEQ ID NO: 68 - DNA, 31 bp, Artificial Sequence
  Synthesized forward primer
ggaattccatatgaacgatctattttcactg

SEQ ID NO: 69 - DNA, 28 bp, Artificial Sequence
  Synthesized reverse primer
gcggatccttaaacagccactaacatgc

SEQ ID NO: 70 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRRHPFTEFPEQDWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 71 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRRHPFTEFPEQEWNDVIAVNQTAVFLVSQAVTRHMVERKAGKVINICSIQSELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 72 - protein, 254 aa, Escherichia coli
MNDLFSLAGKNILITGSAQGIGFLLATGLGKYGAQIIINDITAERAELAVEKLHQEGIQAVAVPFNVTHKHEIDAAVEHIETDIGTIDVLVNNAAIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 73 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIETDIGTIDVLVNNAGIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALVEDVAFTAWLCKRTPAARWGDPQELIGAAVFLSSKASDFVNGHLLFVDGGMLVAL

SEQ ID NO: 74 - protein, 254 aa, Escherichia coli
MNDLFSLAGKNVLITGSAQGIGFLLATGLGKYGAQIIINDILAKRAELAVEKLHQEGIQAVAAPFNVTHKHEIDAAVEHIETDIGTIDVLVNNAAIQRRHPFTEFPEQEWNDVIAVNQTAVFLVSQAVTRHMVGRKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 75 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIETDIGTIDVLVNNAGIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 76 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSMQSELGRDTTTPYAASKRAVKMLTRGMCVELARHNIQVNGIAPGYIKTEMTKALVEDEAFTAWLCKRTPAARWGDPQELIGAAVFLSSKASDFVNGHLLFVDGGMLVAL

SEQ ID NO: 77 - protein, 254 aa, Escherichia coli
MNDLFSLAGKNILITGSAQGIGFLLATGLGKYGAQIIINDITAERAELAVEKLHQEGIQAVAVPFNVTHKHEIDAAVEHIETDIGTIDVLVNNAAIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAAVFLSSRASDFVNGHLLFVDLGMLVAL

SEQ ID NO: 78 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGLLVAL

SEQ ID NO: 79 - protein, 254 aa, Escherichia coli
MNDLFSLAGKNILITGSAQGIGFLMATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSTASDFVNGHLLFVDGGMLVAL

SEQ ID NO: 80 - protein, 254 aa, Escherichia coli
MNDLFSLAGKNILITGSAQGIGFLLATGLGKYGALIIINDITAERAELAVEKLHQEGIQAVAAPFNVTRKHEIDAAVEHIETDIGTIDVLVNNAAIQRRHPFTEFPEQEWNDVIAVNQTGVFLVSQAVTRHMVERKAGKVINICSIQSELGRDTITPYAASKGAVKMLTRGMCVELARHNIQVNGIAPGYLKTEMTKALVEDEAFTAWLCKRTPAARWGDPQELIGAAVFLSSKASDFVNGHLLFVDGGMLVAL

SEQ ID NO: 81 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIEKDIGHIDVLVNNAGIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 82 - protein, 254 aa, Escherichia coli
MNDLFSLAGKNILITGSAQGIGFLLATGLGKYGAQIIINDITAERAELAVEKLHQEGIQAVAVPFNVTHKHEIDAAVEHIETDIGTIDVLVNNAAIQRRHPFTEFPEQEWNEVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 83 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAGIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 84 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQDWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 85 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIEKDIGPIDVLVNNAGIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 86 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDFGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 87 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAGIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALAEDEAFTAWLCKRTPAARWGDPQELIGAAVFLSSKASDFVNGHLLFVDGGMLVAL

SEQ ID NO: 88 - protein, 254 aa, Escherichia coli
MNDLFSLAGKNILITGSAQGIGFLLATGLGKYGAQIIINDITAERAELAVEKLHQEGIQAVAVPFNVTHKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASEFVNGHLLFVDGGMLVAL

SEQ ID NO: 89 - protein, 254 aa, Escherichia coli
MNDLFSLAGKNILITGSAQGIGFLLATGLGKYGAQIIINDITAERAELAVEKLHQEGIQAVAVPFNVTHKHEIDAAVEHIEKDIGPIDVLVNNAGIQRRHPFTEFPEQEWNDVIAVNQTAVFLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 90 - protein, 254 aa, Escherichia coli
MNDLFSLAGKNILITGSAQGIGFLLATGLGKYGAQIIINDITAERAELAVEKLHQDGIQAVAAPFNVTHKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 91 - protein, 254 aa, Escherichia coli
MNDLFSLAGKNILITGSAQGIGFLLATGLGKYGAQIIINDITAERAELAVEKLHQEGIEAVAVPFNVTHKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDDAFTAWLCKRTPAARWGDPQELIGAAVFLSSKASDFVNGHLLFVDGGMLVAL

SEQ ID NO: 92 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 93 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRLHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 94 - protein, 254 aa, Escherichia coli
MNDLFSLAGKNILITGSAQGIGFLLATGLGKYGAQIIINDITAERAEPAVEKLHQEGIQAVAAPFNVTHKHEIDAAVEHIEKDIGPIDVLVNNAGIQRRHPFTEFPEQEWNDVIAVNQTAVSLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 95 - protein, 254 aa, Escherichia coli
MNDLFSLAGKNILITGSAQGIGFLLATGLGKYGAQIIINDIAAERAELAVEKLHQEGIQAVAAPFNVTHKHEIDAAVDHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVYLSSKASDFVNGHLLFVDGGMLVAL

SEQ ID NO: 96 - protein, 254 aa, Escherichia coli
MNDLFSLAGKNILITGSAQGIGFLLATGLGKYGAQIIINDITAERAELAVEKLHQEGIEAVAAPFNVTHKHEIDAAVEHIETDIGTIDVLVNNAAIQRRHPFTEFPEQEWNDVIAVNQTAVFLVSQAVTRHMVERKAGKVINICSIQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDLGMLVAL

SEQ ID NO: 97 - protein, 254 aa, Escherichia coli
MNDLFSLAGKNILITGSAQGIGFLLATGLGKYGAQIIINDITAERAELAVEKLHQEGIEAVAAPFNVTHKHEIDAAVEHIETDIGTIDVLVNNAAIQRRHPFTEFPEQEWNDVIAVNQTAVFLVSQAVTRHMVERKTGKVINICSIQSELGRDTTTPYAASKGAVKMLTRGMCVELARHNIQVNGIAPGYFKTEMTKALVEDEAFTAWLCKRTPAARWGDPQELIGAAVSLSSKASDFVNGHLLFVDGGMLVAL

SEQ ID NO: 98 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSIQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 99 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASEFVNGHLLFVDGGMLVAL

SEQ ID NO: 100 - protein, 254 aa, Escherichia coli
MNDLFSLAGKNILITGSAQGIGFLLATGLGKYGAQIIINDITAERAELAVDKLHQEGIQAVAVPFNVTHKHEIDAAVEHIEKNIGPIDVLVNTAGIQRRFPFTEFPEQEWNEVIAVNQTAVFLVSQAVTRRMVERKAGKVINIGSMQCELGRDTITPYAASKGAVKMLTRGMCVELARHNIQVNGIAPGYFKTEMTKALVEDEAFTAWLCKRTPAARWGDPQELIGAGVFLSSKASDFVNGHLLFVDGGMLVAL

SEQ ID NO: 101 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRRHPFTEFPEQEWNDVIAVNQTAVFLVSQAITRRMVERKAGKVINIGSMQCELGRDTITPYAASKGAVKMLTRGMCVELARHNIQVNGIAPGYFITEMTKALVEDEAFTAWLCKRTPAARWGDPQELIGAAVFLSSKASDFVNGHLLFVDGGMLVAL

SEQ ID NO: 102 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIETDIGTIDVLVNNAAIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALAEDEAFTAWLCKRTPAARWGDPQELIGAAVFLSSKASDFVNGHLLFVDGGMLVAL

SEQ ID NO: 103 - protein, 254 aa, Escherichia coli
MNDLFSLAGKNILITGSAQGIGFLLATGLGKYGAQIIINDITAERAELAVEKLHQEGIEAVAAPFNVTHKHEIDAAVEHIEKDIGPIDVLVNNAGIQRRHPFTEFPEQEWNDVIAVNQTAVFLVSQAVTRHMVERKAGKVINIGSMQCELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDLGMLVAL

SEQ ID NO: 104 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIEKDIGPIDVLVNNASIQRRHPFTEFPEQEWNDVIAVNQTAVFLVSQAVTRHMVERKAGKVINICSMQSELGRDTTTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL

SEQ ID NO: 105 - protein, 254 aa, Escherichia coli
MNDLFSLAGKNILITGSAQGIGFLLATGLGKYGAQIIINDITAERAELAVEKLHQEGIQAVAAPFNVTRKHEIDAAVEHIETDIGTIDVLVNNAAIQRRHPFTEFPEQEWNDVIAVNQTAVFLVSQAVTRHMVERKAGKVINICSIQSELGRDTLTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAV

SEQ ID NO: 106 - protein, 254 aa, Escherichia coli
MNDLFSLAGKNILITGSAQGIGFLLATGLGKYGAQIIINDITAERAELAVEKLHQEGIQAVAAPFNVTHKHEIDAAVEHIEKDIGPIDVLVNNAAIQRRHPFTEFPEQEWNDVIAVNQTAVFLVSQAVTRHMVERKAGKVINICSIQSELGRDTLTPYAASKGAVKMLTRGMCVELARHNIQVNGIAPGYFKTEMTKALVEDEAFTAWLCKRTPAARWGDPQELIGAAVFLSSKASDFVNGHLLFVDGGMLVAL